(19) United States
(12) Patent Application Publication
(10) Pub. No.: US 2019/0388021 A1
(43) Pub. Date: Dec. 26, 2019

(54) WEARABLE SYSTEMS WITH BATTERY-FREE SENSORS

(71) Applicant: Wearifi, Inc., Champaign, IL (US)

(72) Inventors: Joseph M. McLellan, Quincy, MA (US); Brian E. Mayers, Arlington, MA (US)

(73) Assignee: Wearifi, Inc., Champaign, IL (US)

(21) Appl. No.: 16/448,934

(22) Filed: Jun. 21, 2019

Related U.S. Application Data
(60) Provisional application No. 62/730,080, filed on Sep. 12, 2018.
(60) Provisional application No. 62/730,057, filed on Sep. 12, 2018.
(60) Provisional application No. 62/688,765, filed on Jun. 22, 2018.

(51) Int. Cl. A61B 5/00

(52) U.S. Cl. CPC A61B 5/443; A61B 5/0002; A61B 5/486; G01L 19/0092; A61B 5/6802; A61B 5/445; A61B 5/742

(57) ABSTRACT

The instant disclosure is directed to wearable dermatological systems with battery-free sensors. A wearable system may comprise a wireless, battery-free sensor configured to detect one or more characteristic features of a wearer's skin and communicate information relating to the one or more characteristic features of the wearer's skin to a reader. The reader may be coupled to the wireless, battery-free sensor. The reader may also be configured to collect the information related to the detected one or more characteristic features of the wearer's skin. The wearable system may be configured to measure the hydration level of a user's skin. A kit may also comprise the wearable system and one or more products.

### CROSS-REFERENCE TO RELATED APPLICATIONS

This application claims priority to and benefit of U.S. Provisional Application Ser. No. 62/688,765, filed Jun. 22, 2018, entitled “Wearable Systems with Battery-Free Sensors,” U.S. Provisional Application Ser. No. 62/730,057, filed Sep. 12, 2018, entitled “Wearable Dermatological Systems with Battery-Free Sensors,” and U.S. Provisional Application Ser. No. 62/730,080, filed Sep. 12, 2018, entitled “Wearable Systems with Battery-Free Sensors,” each of which is hereby incorporated herein by reference in its entirety.

### BACKGROUND

Wearable systems for detecting and measuring the environment of a person, animal, plant, or object can be useful for clinical, agricultural, and environmental purposes. Digital electronic sensing technology provides an accurate and versatile means for determining one or more features of an environment, particularly a dermatological environment. However, traditional approaches to detection and measurement require bulky, expensive devices comprising integrated circuits, detectors, batteries, memory, display panels and power management systems. Such systems are not always practical or cost-effective. In addition, incorporating such systems into garments and other wearable items may interfere with the utility and comfort of those systems. Therefore, there exists a need for wearable systems with wireless, battery-free environmental sensors.

### SUMMARY

The instant disclosure is directed to wearable systems with battery-free sensors. In one embodiment, a wearable system may comprise a wireless and battery-free sensor configured to detect one or more characteristic features of a wearer's skin, and to communicate information relating to the one or more characteristic features of the wearer's skin to a reader. In an embodiment, the reader may be coupled to the wireless and battery-free sensor. In some embodiments, the reader may also be configured to collect information relating to the detected one or more characteristic features of the wearer's skin. In certain embodiments, the detected one or more characteristic features of the wearer's skin may comprise, for example, a hydration level of the wearer's skin. In an embodiment, a kit may comprise such a wearable system, and a product.

In an embodiment, a wearable system may comprise a wireless and battery-free sensor configured to detect one or more characteristic features of a wearer's skin. In an embodiment, the wearable system may further include a transponder, transmitter, or transducer coupled to the wireless and battery-free sensor and configured to communicate information relating to the detected one or more characteristic features of the wearer's skin to a device. Further embodiments of the instant disclosure are described herein.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

As used herein, the term “comprising” means “including, but not limited to.” In embodiments or claims where the term “comprising” is used as the transition phrase, such embodiments can also be envisioned with replacement of the term “comprising” with the terms “consisting of” or “consisting essentially of.”

The terms “flexible” and “bendable” are used synonymously in the present description and refer to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. As used herein, some, but not necessarily all, flexible structures are also stretchable. A variety of properties provide flexibility in structures (e.g., device components), including materials properties such as a low modulus, bending stiffness and flexural rigidity, physical dimensions such as small average thicknesses (e.g., less than 100 microns, optionally less than 10 microns and optionally less than 1 micron) and device geometries such as thin film and mesh geometries.

As used herein, “stretchable” refers to the ability of a material, structure, device or device component to be strained without undergoing fracture. In an exemplary embodiment, a stretchable material, structure, device or device component may undergo strain larger than 0.5% without fracturing, for some applications strain larger than 1% without fracturing and for yet other applications strain larger than 3% without fracturing. As used herein, many stretchable structures are also flexible. Some stretchable structures (e.g., device components) are engineered to be able to undergo compression, elongation and/or twisting so as to be able to deform without fracturing. Stretchable structures include thin film structures comprising stretchable materials, such as elastomers; bent structures capable of elongation, compression and/or twisting motion; and structures having an island-bridge geometry.

Stretchable device components include structures having stretchable interconnects, such as stretchable electrical interconnects.

As used herein, “functional layer” refers to a device-containing layer that imparts some functionality to the device. For example, the functional layer may be a thin film such as a semiconductor layer. Alternatively, the functional layer may comprise multiple layers, such as multiple semiconductor layers separated by support layers. The functional layer may comprise a plurality of patterned elements, such as interconnects running between device-receiving pads or islands. The functional layer may be heterogeneous or may have one or more properties that are inhomogeneous. An “inhomogeneous property” refers to a physical parameter that can spatially vary, thereby effecting the position of the neutral mechanical surface (NMS) within the multilayer device.

As used herein, “semiconductor” refers to any material that is an insulator at a low temperature, but which has an appreciable electrical conductivity at temperatures of approximately 300 Kelvin. In the present description, use of the term “semiconductor” is intended to be consistent with use of this term in the art of microelectronics and electronic devices. Useful semiconductors include those comprising element semiconductors, such as silicon, germanium and diamond, and compound semiconductors, such as group IV compound semiconductors such as SiC and SiGe, group lll-V semiconductors such as AlSb, AIAs, Aln, AIP, BN, GaSb, GaAs, GaN, GaP, InSb, InAs, InN, and InP, group ll I-V ternary semiconductors alloys, such as AlxGai-xAs, group ll-VI semiconductors, such as CsSe, CdS, CdTe, ZnO, ZnSe, ZnS, and ZnTe, group I-VII semiconductors, such as CuCI, group IV-VI semiconductors, such as PbS, PbTe and SnS, layer semiconductors, such as Pbl2, M0S2 and GaSe, and oxide semiconductors, such as CuO and CU2O. The term semiconductor includes intrinsic semiconductors and extrinsic semiconductors that are doped with one or more selected materials, including semiconductors having p-type doping materials and n-type doping materials, to provide beneficial electronic properties useful for a given application or device. The term semiconductor includes composite materials comprising a mixture of semiconductors and/or dopants. Specific semiconductor materials useful for some embodiments include, but are not limited to, Si, Ge, SiC, AIP, AIAs, AlSb, GaN, GaP, GaAs, GaSb, InP, InAs, GaSb, InP, InAs, InSb, ZnO, ZnSe, ZnTe, CdS, CdSe, ZnSe, ZnTe, CdS, CdSe, CdTe, HgS, PbS, PbSe, PbTe, AIGaAs, AllnAs, AlInP, GaAsP, GalnAs, GalnP, AIGaAsSb, AIGalnP, and GalnAsP. Porous silicon semiconductor materials are useful for applications of aspects described herein in the field of sensors and light emitting materials, such as light emitting diodes (LEDs) and solid-state lasers. Impurities of semiconductor materials are atoms, elements, ions and/or molecules other than the semiconductor material(s) themselves or any dopants provided to the semiconductor material. Impurities are undesirable materials present in semiconductor materials, which may negatively affect the electronic properties of semiconductor materials, and include but are not limited to oxygen, carbon, and metals including heavy metals. Heavy metal impurities include, but are not limited to, the group of elements between copper and lead on the periodic table, calcium, sodium, and all ions, compounds and/or complexes thereof.

As used herein, “coincident” refers to the relative position of two or more objects, planes or surfaces, for example a surface such as a neutral mechanical surface (NMS) or neutral mechanical plane (NMP) that is positioned within or is adjacent to a layer, such as a functional layer, substrate layer, or other layer. In an embodiment, a NMS or NMP is positioned to correspond to the most strain-sensitive layer or material within the layer. “Proximate” refers to the relative position of two or more objects, planes or surfaces, for example a NMS or NMP that closely follows the position of a layer, such as a functional layer, substrate layer, or other layer while still providing desired flexibility or stretchability without an adverse impact on the strain-sensitive material physical properties. In general, a layer having a high strain sensitivity, and consequently being prone to being the first layer to fracture, is located in the functional layer, such as a functional layer containing a relatively brittle semiconductor or other strain-sensitive device element. A NMS or NMP that is proximate to a layer need not be constrained within that layer, but may be positioned proximate or sufficiently near to provide a functional benefit of reducing the strain on the strain-sensitive device element when the device is folded.

As used herein, “strain-sensitive” refers to a material that fractures or is otherwise impaired in response to a relatively low level of strain. In an aspect, the NMS is coincident or proximate to a functional layer. In an aspect, the NMS is coincident to a functional layer, referring to at least a portion of the NMS located within the functional layer that contains a strain-sensitive material for all lateral locations along the NMS. In an aspect, the NMS is proximate to a functional layer, wherein although the NMS may not be coincident with the functional layer, the position of the NMS provides a mechanical benefit to the functional layer, such as substantially lowering the strain that would otherwise be exerted on the functional layer but for the position of the NMS. For example, the position of a proximate NMS is optionally defined as the distance from the strain-sensitive material that provides an at least 10%, 20%, 50% or 75% reduction in strain in the strain-sensitive material for a given folded configuration, such as a device being folded so that the radius of curvature is on the order of the millimeter or centimeter scale. In another aspect, the position of a proximate NMS can be defined in absolute terms such as a distance from the strain-sensitive material, such as less than several mm, less than 2 mm, less than 10 μm, less than 1 μm, or less than 100 nm. In another aspect, the position of a proximate layer is defined relative to the layer that is adjacent to the strain-sensitive material, such as within 50%, 25% or 10% of the layer closest to the strain-sensitive-containing layer. In an aspect, the proximate NMS is contained within a layer that is adjacent to the functional layer.

As used herein, “sensing” refers to detecting the presence, absence, amount, magnitude or intensity of a physical and/or chemical property. Useful device components for sensing include, but are not limited to electrode elements, chemical or biological sensor elements, pH sensors, temperature sensors, strain sensors, mechanical sensors, position sensors, optical sensors, environmental sensors, and capacitive sensors.

As used herein, the term “polymer” includes homopolymers, or polymers consisting essentially of a single repeating monomer subunit. The term “polymer” also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components include, but are not limited to, plastics, elastomers, thermoplastic elastomers, elastoplastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

As used herein, “elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. An “elastomeric layer” refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

As used herein, the term “conformable” refers to a device, material or substrate which has a bending stiffness that is sufficiently low to allow the device, material or substrate to adopt a contour profile desired for a specific application, for example a contour profile allowing for conformal contact with a surface having a non-planar geometry such as a surface with relief features or a dynamic surface (e.g. changes with respect to time).

As used herein, “conformal contact” refers to contact established between a device and a receiving surface. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface.

As used herein, high Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably about 10 times larger for some applications, more preferably about 100 times larger for other applications, and even more preferably about 1000 times larger for yet other applications. In an embodiment, a low modulus layer has a Young's modulus less than 100 MPa, optionally less than 10 MPa, and optionally a Young's modulus selected from the range of 0.1 MPa to 50 MPa. In an embodiment, a high modulus layer has a Young's modulus greater than 100 MPa, optionally greater than 10 GPa, and optionally a Young's modulus selected from the range of 1 GPa to 100 GPa. In an embodiment, a device of the invention has one or more components having a low Young's modulus. In an embodiment, a device of the invention has an overall low Young's modulus. In some embodiments, “low modulus” refers to materials having a Young's modulus less than or equal to 10 MPa, less than or equal to 20 MPa or less than or equal to 1 MPa.

As used herein, “bending stiffness” is a mechanical property of a material, device or layer describing the resistance of the material, device or layer to an applied bending movement. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

As used herein, “lateral dimensions” refer to physical dimensions of a structure such as a wearable system or component thereof. For example, lateral dimensions may refer to one or more physical dimensions oriented orthogonal to axes extending along the thickness of a structure, such as the length, the width, the radius or the diameter of the structure. Lateral dimensions are useful for characterizing the area of an electronic system or component thereof, such as characterizing the lateral area footprint of a system corresponding to a two dimensional area in a plane or a surface positioned orthogonal to axes extending along the thickness of the structure.

As used herein, “ambient parameter” refers to a condition, state or property experienced by a monitoring system, such as an environmental condition, state or property. In an embodiment, for example, an ambient parameter is capable of being detected, monitored and/or converted into an electric signal. Exemplary ambient parameters include but are not limited to incident electromagnetic radiation, nuclear radiation, temperature, incident ionizing radiation, heat, movement (e.g., acceleration), strain, pollution (gaseous, liquid and particulate), sound (acoustic waves), magnetic forces, humidity, and moisture.

As used herein, the term “measurement” refers to generation of a signal indicative of an ambient parameter. “Readout” refers to transfer or transmission of the measured signal, or a signal derived from the measured signal, for example to an external device such as a computer or mobile device. In an embodiment of the present invention, the measurement and readout functions of a monitoring system are independently powered.

As used herein, the expression “long-term conformal integration” refers to the capability of the present systems to establish and maintain conformal contact with a wearable system for at least 3 hours, optionally at least 1 day or at least 1 month, without undergoing delamination or other degradation sufficient to impair electronic or photonic performance.

As used herein, the expression “conformal integration with the wearable system without inflammation or immune response” refers to the capability of the present systems to establish conformal contact with a wearable system without causing an observable inflammation or immune response from the user.

As used herein, the expression “conformal integration with the wearable system without substantially changing the exchange of heat and fluids” refers to the capability of the present systems to establish conformal contact with a wearable system without changing the amount of heat and fluids absorbed or released from the wearable system at the mounting site by a factor greater than 75%, optionally greater than 25%, relative to the wearable system without the mounted device.

### Wireless, Battery-Free Sensors

The wireless, battery-free sensors described below are exemplary embodiments of wireless, battery-free sensors that may be incorporated into wearable systems. However, other wireless, battery-free sensors known in the art are not excluded from use in the wearable systems described herein. For example, wireless, battery-free sensors are described in WO 2016/196675 and WO 2016/196673, each of which is incorporated herein by reference in its entirety.

In some embodiments, the wireless, battery-free sensors described herein implement high performance, and optionally flexible, device components having miniaturized formats in device architectures that minimize adverse physical effects to tissue, garments, and the like. In some embodiments, the invention provides complementary garment or object mounting strategies providing for mechanically robust and/or long-term integration of the present devices. Devices of the invention are versatile and support a broad range of applications for sensing, actuating and communication including applications for near field communication, for example, for electronic communications and biometric sensing.

In some embodiments, the wireless, battery-free sensors may comprise: (i) a substrate having an inner surface and an outer surface; and (ii) an electronic device comprising one or more inorganic and/or organic components supported by the outer surface of the substrate; wherein the electronic device has a thickness less than or equal to 5 millimeters, optionally less than 1 millimeter, and has lateral dimensions small enough to provide long-term conformal integration via direct or indirect contact with the wearable system without substantial delamination.

In other embodiments, the wireless, battery-free sensors may comprise: (i) a substrate having an inner surface and an outer surface; and (ii) an electronic device comprising one or more inorganic components, organic components or a combination of inorganic and organic components supported by the outer surface of the substrate; wherein the electronic device has a thickness less than or equal to 10 millimeters, optionally less than 5 millimeters or 1 millimeter, and has lateral dimensions small enough to provide conformal integration with the wearable system without inflammation or immune response from the user.

In still other embodiments, the wireless, battery-free sensors may comprise: (i) a substrate having an inner surface and an outer surface; and (ii) an electronic device comprising one or more inorganic components, organic components or combination of inorganic and organic components supported by the outer surface of the substrate; wherein the electronic device has a thickness less than or equal to 10 mm, optionally less than 5 mm or 1 mm, and has lateral dimensions small enough to provide conformal integration with the wearable system without substantially changing the exchange of heat and fluids from the wearable system upon which the system is mounted.

Miniaturized thickness and lateral dimensions are significant in some embodiments, for example, wherein the wireless, battery-free sensor is characterized by a maximum thickness less than 2 mm, optionally less than 125 microns, less than 0.1 microns or less than 0.05 microns, and/or wherein the electronic device is characterized by an area of less than 2 cm², optionally less than 0.5 cm² or less than 0.1 cm². In an embodiment, the wireless, battery-free sensor is directly supported by and in physical contact with the wearable system or indirectly supported by the wearable system, for example, via one or more intermediate components provided in between the wireless, battery-free sensor and the wearable system.

In certain embodiments, the wireless, battery-free sensors may comprise: (i) a substrate having an inner surface and an outer surface; and (ii) an electronic device comprising one or more inorganic components, organic components or combination of inorganic and organic components supported by the outer surface of the substrate; wherein the electronic device is capable of establishing conformal integration with the wearable system, and wherein the electronic device undergoes a transformation upon an external stimulus or an internal stimulus; wherein the transformation provides a change in function of the system from a first condition to a second condition. Systems of this aspect may be compatible with a range of external and/or internal stimuli, including movement of the system, tempering with the system, a physical, chemical or electromagnetic change of the system, change in a measured signal or property, change in an ambient parameter and combinations of these. In an embodiment, the transformation provides the change in function of the device from a first condition of operability to a second condition of inoperability. In an embodiment, the transformation is induced upon removal or attempted removal of the system from a mounting position on the wearable system. In an embodiment, the transformation is induced by a physical change, a chemical change, a thermal change or electromagnetic change of the system or a component thereof. In an embodiment, the transformation is induced by physical breakage of a component of the system (e.g., breakage of an active component, breakage of an electronic interconnect, breakage of the substrate, breakage of a barrier layer or encapsulating layer, etc.), a physical deformation of a component of the system (e.g. deformation of an active component, deformation of an electronic interconnect, deformation of the substrate, etc.), a change in physical conformation of the system (e.g., change in contour, a change in curvature, etc.), or removal of a barrier or encapsulation layer of the system, for example, such that resulting exposure to the environment induces a change. In an embodiment, the transformation is induced by a change in a value of a measured device property (e.g., state of strain, antenna property), a measured physiological property of the tissue or subject (e.g., temperature, pH level, glucose, pulse oximetry, heart rate, respiratory rate, blood pressure, peripheral capillary oxygen saturation (SpO2)) or measured ambient property (e.g., temperature, electromagnetic radiation, etc.). In an embodiment, the transformation is induced by a positional change (e.g., movement of the system) or a temporal change (e.g., upon elapse of a preselected time period).

In other embodiments, the wireless, battery-free sensors may comprise: (i) a substrate having an inner surface and an outer surface; wherein the inner surface of the substrate is for establishing contact with a wearable system; and (ii) an electronic device comprising one or more inorganic components, organic components or a combination of inorganic and organic components; wherein each of the inorganic components is supported by the outer surface and independently positioned within 20 mm, optionally 16 mm, or 10 mm, or 1 mm, of an edge of the substrate (e.g., perimeter edge or edge of a cut-out region positioned away from the perimeter); wherein the wearable system-mounted electronic device has lateral dimensions less than or equal to 20 mm, optionally for some applications less than or equal to 16 mm, and a thickness less than or equal to 5 mm, optionally for some applications less than or equal to 10 mm. In an embodiment of this aspect, the electronic system is directly supported by and in physical contact with the wearable system or indirectly supported by the wearable system, for example, via one or more intermediate components provided in between the system and the wearable system.

In some embodiments, for example, the wireless, battery-free sensors may have lateral dimensions selected from the range of 5 mm to 20 mm. In some embodiments, for example, the system has thickness dimensions selected from the range of 0.125 mm to 5 mm, or 0.005 mm to 5 mm. In some embodiments, for example, the system is characterized by a footprint/contact area of 10 mm² to 500 mm², or 20 mm² to 350 mm², or 30 mm² to 150 mm², in some embodiments the system is characterized by a footprint/contact area greater than 25 mm² or greater than 20 mm². In some embodiments, for example, the system has a tapered thickness from the center to outer edge. In some embodiments, a taper of not less than 5 degrees, or not less than 10 degrees, from the center of the system to the outer edge reduces or prevents delamination. In some embodiments, the system is symmetrically or asymmetrically tapered from the center to the outer edges. In some embodiments, for example, the system has a shape selected from the group consisting of elliptical, rectangular, circular, serpentine and irregular shapes. In some embodiments, for example, the system is characterized by component lateral dimensions selected from 4 mm to 16 mm.

In some embodiments, the wireless, battery-free sensors may be designed to reduce or prevent delamination, for example, via having a tapered geometry. In an embodiment, for example, a portion of, or all, intersecting outer surfaces are joined radially at an angle to reduce or prevent delamination. In an embodiment, for example, the system is characterized by a gradual reduction of thickness in a range equal to or less than the center of the device to the outer surface to reduce or prevent delamination. In an embodiment, for example, a thickness at an edge, such as an outer edge of the system or an edge of an aperture of the system, is at least 2 times, or at least 5 times, or at least 10 times, less than a thickness at a center (or mid-point between edges) of a system. In an embodiment, a thickness of the overall system decreases substantially asymptotically from a mid-point of the system to an edge, such as an outer edge of the system or an edge of an aperture of the system.

In some embodiments, the wireless, battery-free sensors may be waterproof, for example, by encapsulation or packaging, with a biopolymer, a thermoset polymer, a rubber, an adhesive tape, plastic or any combination of these. For example, in embodiments, the system comprises an encapsulation layer or other waterproofing structure comprising polyimide, parylene, vinyl, acrylic, polydimethylsiloxane (PDMS), polyurethane, vinyl, polystyrene, polymethyl methacrylate (PMMA) or polycarbonate.

In embodiments, the inorganic and/or organic components of the wireless, battery-free sensors are selected from inorganic and/or organic semiconductor components, metallic conductor components and combinations of inorganic semiconductor components and metallic conductor components. In an embodiment, for example, each of the inorganic components is independently positioned within 10 mm, optionally within 1 mm, of an edge of the perimeter of the substrate. In an embodiment, each of the inorganic components is independently positioned within 10 mm, and in some embodiments less; e.g. optionally within 1 mm, of an edge of an aperture in the substrate. In an embodiment, each of the inorganic components is independently characterized by a shortest distance to an edge of the substrate, wherein an average of the shortest distances for the inorganic components is equal to or less than 10 mm, optionally equal to or less than 1 mm.

The wireless, battery-free sensors described herein exploit overall size miniaturization to achieve a mechanically robust interface with a wearable system surface without generating stresses or strains adversely impacting performance and/or to minimize adverse physical effects to garments and/or other objects. In embodiments, for example, the wireless, battery-free sensors may have a lateral area footprint less than or equal to 500 mm², optionally less than or equal to 315 mm², or selected from the range of 1 mm² to 500 mm² and optionally selected from the range of 1 mm² to 315 mm². In embodiments, the wireless, battery-free sensors may have an average thickness selected from the range of 5 microns to 5 mm, optionally 12 microns to 1 mm, optionally 50 microns to 90 microns, or, for example, greater than 50 microns. In an embodiment, the wireless, battery-free sensors may have an overall maximum thickness less than 0.2 mm and at least one region having a thickness selected from the range of 0.05 mm to 0.1 mm. For example, a region of the wireless, battery-free sensors comprising a relatively thick component, such as an NFC chip or an LED, may provide a thickness less than 0.2 mm and a region of the wireless, battery-free sensors comprising a relatively thin component, such as only substrate, may provide a thickness selected from the range of 0.05 mm to 0.1 mm, or a thickness of less than 0.09 mm, or less than 0.07 mm.

The wireless, battery-free sensors described herein may integrate thin, flexible functional components and substrates to provide sufficient mechanical compliance to achieve a conformal interface at the mounting site for a tissue surface. Advantages of mechanically flexible systems of the invention include the ability to conform to complex contoured wearable systems.

In embodiments, the wireless, battery-free sensors may have an average modulus selected from the range of 10 kPa to 100 GPa, or greater than 10 kPa, optionally greater than 100 MPa. In embodiments, the tissue mounted electronic system has a flexural rigidity selected from the range of 0.1 nN m to 1 N m. In an embodiment, the wireless, battery-free sensors may have a net bending stiffness of greater than 0.1 nN m, optionally for some applications greater than 10 nN m, and optionally for some applications greater than 1000 nN m. In some embodiments, for example, one or more mechanical properties of the device, such as average modulus, flexural rigidity or bending stiffness, are matched to properties of the wearable system at the mounting site; e.g., within a factor of 5. In embodiments, the wireless, battery-free sensors may have an adhesion strength selected from the range of 1 N/25 mm to 50 N/25 mm, or the tissue mounted electronic system has an adhesion strength greater than 50 N/25 mm, or greater than 60 N/25 mm. In some embodiments, peel adhesion can be tuned for specific applications after 20 minutes at room temperature.

The wireless, battery-free sensors described herein include multilayer devices, for example, wherein functional layers having electronically and/or optoelectronically functional device components are separated from each other by structural layers, such as electrically insulating or supporting layers or coatings. In embodiments, the wireless, battery-free sensors may have a multilayer geometry comprising a plurality of functional layers, supporting layers, encapsulating layers, planarizing layers or any combination of these. In embodiments, the wireless, battery-free sensors may have a shape selected from the group consisting of elliptical, rectangular, circular, serpentine and/or irregular. In an embodiment, the shape is characterized by an aspect ratio of a lateral dimension to thickness less than 10,000 or optionally for some embodiments selected from the range of 5000 to 3.

Substrates having a range of physical and chemical properties are useful in the wireless, battery-free sensors described herein. The invention includes substrates having functionality as an electrical insulator, an optically transparent layer, an optical filter and/or a mechanically supporting layer. In embodiments, the inner surface of the substrate has an area for establishing the conformal contact with the tissue surface less than or equal to 315 mm², or selected from the range of 19 mm² to 315 mm². In an embodiment, the substrate has a perforated geometry including a plurality of apertures extending through the substrate. In an embodiment, the substrate is discontinuous. In an embodiment, the apertures allow passage of gas and fluid from the tissue through the device, in some embodiments, the apertures allow transport of fluid away from the tissue surface. In an embodiment, each of the apertures is independently characterized by lateral dimensions selected from the range of 12 microns to 5 mm, or 25 microns to 1 mm, or 50 microns to 500 microns. In an embodiment, perforations are distributed in the substrate with a pitch selected from the range of 4 mm to 0.2 mm, or 2 mm to 0.5 mm. In an embodiment, the perforations are openings, such as circular openings, having average diameters greater than 0.1 mm and less than 2 mm, or greater than 0.2 mm and less than 1 mm. In an embodiment, the substrate has an areal density of the apertures selected from the range of one per cm² to one hundred per cm². In an embodiment, the apertures are provided in a substantially spatially uniform distribution across the substrate. In an embodiment, the apertures provide an overall mesh geometry of the substrate. In an embodiment, the apertures provide a porosity of the substrate equal to or greater than 0.01%, optionally for some embodiments equal to or greater than 0.1%, or equal to or greater than 1%, or equal to or greater than 10%. In an embodiment, a perforated or discontinuous substrate comprises at least 0.01% open area, at least 0.1% open area, at least 0.5% open area, at least 1% open area, at least 5% open area, or at least 10% open area. In an embodiment, each of the apertures is independently characterized by a cross sectional area selected from the range of 100 μm² to 1 cm², or 200 μm² to 1 mm², or 500 μm² to 0.5 mm².

In embodiments, the substrate is a flexible substrate or a stretchable substrate. In an embodiment, the substrate is characterized by an average modulus selected from the range of 10 kPa to 100 GPa, or greater than 10 kPa, optionally for some applications greater than 10 kP. In an embodiment, the substrate is characterized by an average thickness selected from the range of 12 microns to 5 mm, 25 microns to 1 mm, or 50 microns to 90 microns, and in some embodiments, greater than 500 microns, optionally for some embodiments, greater than 1000 microns.

In an embodiment, the substrate comprises one or more thin films, coatings or both. For example, in some embodiments, a coating or thin film is provided directly on the electronic device or component thereof, and in some embodiments, in direct physical contact. In some embodiments, however, the coating or thin film is provided on an intermediate structure positioned between the electronic device and the coating or film. In embodiments, the substrate comprises an inorganic polymer, an organic polymer, a plastic, an elastomer, a biopolymer, a thermoset polymer, a rubber, an adhesive tape or any combination of these. For example, in embodiments, the substrate comprises polyimide polydimethylsiloxane (PDMS), polyurethane, cellulose paper, cellulose sponge, polyurethane sponge, polyvinyl alcohol sponge, silicone sponge, polystyrene, polymethyl methacrylate (PMMA) or polycarbonate.

A range of functional electronic device components and device integration strategies are compatible with the present systems, thereby supporting expansive applications in wearable electronics. In an embodiment, for example, the system further comprises one or more encapsulating layers or coatings for encapsulating the electronic device. In embodiments, the electronic device is a rigid device, a semi-rigid device, a flexible electronic device or a stretchable electronic device. In embodiments, for example, each of the one or more inorganic or organic components independently comprises one or more thin films, nanoribbons, microribbons, nanomembranes or micromembranes. In an embodiment, the one or more inorganic or organic components independently comprise a single crystalline inorganic semiconductor material.

In an embodiment, for example, the one or more inorganic or organic components of the wireless, battery-free sensors independently have a thickness selected from the range of 5 microns to 5000 microns, optionally for some applications 50 microns to 100,000 microns, optionally for some applications the range of 50 microns to 2000 microns. In an embodiment, for example, the one or more inorganic or organic components independently have a thickness greater than 5 microns and optionally for some embodiments a thickness greater than 50 microns. In an embodiment, the one or more inorganic or organic components are independently characterized by a curved geometry, for example, a bent, coiled, interleaved or serpentine geometry. In an embodiment, the one or more inorganic or organic components are characterized by one or more island and bridge structures.

In embodiments, the wireless, battery-free sensors have a multilayer geometry comprising a plurality of functional layers, barrier layers, supporting layers and encapsulating layers. In an embodiment, the wireless, battery-free sensors are provided proximate to a neutral mechanical surface of the system. In an embodiment, for example, the wireless, battery-free sensors may include, for example, sensors selected from the group consisting of an optical sensor, an electrochemical sensor, a chemical sensor, a mechanical sensor, a pressure sensor, an electrical sensor, a magnetic sensor, a strain sensor, a temperature sensor, a heat sensor, a humidity sensor, a motion sensor (e.g., accelerometer, gyroscope), a color sensor (colorimeter, spectrometer), an acoustic sensor, a capacitive sensor, an impedance sensor, a biological sensor, an electrocardiography sensor, an electromyography sensor, an electroencephalography sensor, an electrophysiological sensor, a photodetector, a particle sensor, a gas sensor, an air pollution sensor, an environmental sensor, an environmental sensor and an imaging device.

In an embodiment, the wireless, battery-free sensors comprise one or more actuators or a component thereof, for example, actuators or a component thereof generating electromagnetic radiation, optical radiation, acoustic energy, an electric field, a magnetic field, heat, a RF signal, a voltage, a chemical change or a biological change. In embodiments, the one or more actuators or a component thereof are selected from the group consisting of a heater, an optical source, an electrode, an acoustic actuator, a mechanical actuator, a microfluidic system, a MEMS system, a NEMS system, a piezoelectric actuator, an inductive coil, a reservoir containing a chemical agent capable of causing a chemical change or a biological change, a laser, and a light emitting diode.

In embodiments, the wireless, battery-free sensors comprise one or more energy storage systems or a component thereof, for example, energy storage systems or components thereof selected from the group consisting of an electrochemical cell, a fuel cell, a photovoltaic cell, a wireless power coil, a thermoelectric energy harvester, a capacitor, a super capacitor, a primary battery, a secondary battery and a piezoelectric energy harvester.

In embodiments, the wireless, battery-free sensors comprise one or more communication systems or a component thereof, for example, communication systems or components thereof selected from the group consisting of a transmitter, a receiver, a transceiver, an antenna, and a near field communication device.

In embodiments, the wireless, battery-free sensors comprise one or more coils, for example, inductive coils or near-field communication coils. In an embodiment, each of the near-field communication coils independently has a diameter selected from the range of 50 microns to 20 mm. In an embodiment, for example, each of the near-field communication coils independently has an average thickness selected from the range of 1 micron to 5 mm, 1 micron to 500 microns, 1 micron to 100 microns, 5 microns to 90 microns, or 50 microns to 90 microns. In an embodiment, for example, each of the near-field communication coils changes by less than 50%, and optionally changes by less than 20%, upon changing from a planar configuration to a bent configuration characterized by a radius of curvature selected from the range of 1 mm to 20 mm. In an embodiment, each of the near-field communication coils is characterized by a Q factor greater than or equal to 3. In an embodiment, the one or more coils are at least partially encapsulated by the substrate or one or more encapsulation layers. In embodiments, for example, the one or more coils have a geometry selected from the group consisting of an annulus or an elliptical annulus. In an embodiment, the tissue mounted system of the invention comprises at least two layered coils, wherein the coils are separated by a dielectric layer.

In some embodiments, the transfer of information to and/or from the system is done wirelessly, for example, through ISO standards such as ISO14443 for proximity contactless cards, ISO15693 for vicinity contactless cards, ISO18000 set of standards for RFIDs and EPC global Class 1 Gen 2 (=18000-6C).

In some embodiments, the wireless, battery-free sensors comprise one or more LED components, for example, to provide an indication of device functionality or for aesthetics. In an embodiment, for example, the system includes one or more LED components designed to remain on after being removed from a reader.

In some embodiments, for privacy, the wireless, battery-free sensors comprise a devoted chip that stores an encrypted identification number that is unique to each individual device. In addition, the chip has action-specific security codes that can change constantly or intermittently. The encrypted device number helps keep patient health-care information private. Clinicians, hospital management, and insurance providers are the only users with access to the information. In case of emergency, hospital personnel can quickly locate missing patients and/or observe patient vital signs.

### Wearable Systems

The wearable systems described herein incorporate of wireless, battery-free sensors into wearable components in a way that allows users and/or subjects to wear these systems without needing to charge them, keep them away from elements such as water or sand, or plug them in. Instead, the wearable systems described herein incorporate miniaturized wireless, battery-free sensors that allow for comfort, ease of use, flexibility, and versatility. The function of a wearable system as described herein would not be disrupted by conditions such as sweat, sand, water, soap, machine washing, rain, nearby electronic systems, and the like.

In some embodiments, a wearable system may comprise a wireless and battery-free sensor as described above. In some embodiments, the wireless and battery-free sensor may be configured to detect one or more characteristic features of a wearer's skin, and may also be configured to reflect and/or communicate the information relating to the one or more characteristic features of the wearer's skin to a reader. The reader may be coupled to the sensor. In certain embodiments, the reader may comprise a display. In some embodiments, the display may be configured to display the information relating to the detected one or more characteristic features of the wearer's skin. In an embodiment, the display may comprise a graphical user interface (GUI). In other embodiments, the reader may comprise an indicator, such as an LED light, for example.

In some embodiments, the one or more characteristic features of the wearer's skin may include a hydration level of the wearer's skin at one or more depths. The one or more depths may range, for example, from about 100 nm to about 20 mm under a surface of the wearer's skin. The one or more depths may include, for example, about 100 nm, about 1,000 nm, about 10,000 nm, about 0.1 mm, about 1 mm, about 10 mm, about 20 mm, or any range between any two of these values, including endpoints. In certain embodiments, the reader may be configured to measure the hydration level of a wearer's skin.

In certain embodiments, the wearable system also comprises a controller configured to suggest that a user perform at least one function in the event that a hydration level deviates from a predetermined target value. The at least one function may include, for example, applying a product to the wearer's skin. The product may be, for example, a sunscreen, a soap, a lotion, a cream, a spray, a gel, a dermatological product, a garment, or combinations thereof. In some embodiments, the product may be configured to induce dry skin. In other embodiments, the product may be configured to treat dry skin. The at least one function may also include, for example, taking an oral medication or an oral supplement.

In some embodiments, the controller may be configured to suggest that the user perform the at least one function by issuing a signal to the wearer (i.e., user) of the wearable system. The signal may include, for example, an audible signal, a visual signal, or a combination thereof. In certain embodiments, the controller may be configured to issue the signal by displaying at least some of the information relating to the detected one or more characteristic features of the wearer's skin on a display screen of a communications device. In certain embodiments, the controller may be a cloud-based controller. In some embodiments, the controller may be coupled to an application software configured to receive the information relating to the one or more characteristic features of the wearer's skin, and to display the suggestion of performing the at least one function. In some embodiments, the reader of the wearable system may comprise a display, and the display may be configured to display the information relating to the detected one or more characteristic features of the wearer's skin. The display may also be configured to display the suggestion that the user perform the at least one function.

In some embodiments, a wearable system may further comprise a wireless, battery-free sensor as described herein, the sensor configured to detect radiation incident on the wearer's skin (a “radiation sensor”). The radiation sensor may be configured to detect radiation at one or more radiation wavelengths. In some embodiments, the one or more radiation wavelengths include at least one of wavelengths ranging from 180 nm to 1200 nm, wavelengths ranging from 315 nm to 400 nm, wavelengths ranging from 280 nm to 315 nm, wavelengths ranging from 280 nm to 400 nm, and wavelengths ranging from 400 nm to 800 nm. In some embodiments, the detected radiation may be X-ray radiation. In other embodiments, the detected radiation may be any other type of radiation known in the art, including UV radiation. In certain embodiments, the radiation sensor may comprise a dosimeter.

In certain embodiments, the wearable system may be implemented in a wearable article such as a garment or object. The wearable system may be implemented in, for example, a hat, a shirt, a jacket, pants, a visor, shorts, a swimsuit, a hospital gown, a smock, hospital scrubs, a blouse, a dress, a skirt, a helmet, a glove, a mitten, an undergarment, footwear, eyewear, a tie, a necklace, an earring, a watch, a bracelet, a band, a hair accessory, a ring, a bag, a backpack, a belt, a wearable accessory, a zipper, a button, a flap, a snap, a patch, a piece of trim, or a combination thereof.

In some embodiments, the wearable system may be implemented on a portion of a skin surface of a wearer of the wearable system. In other embodiments, the wearable system may be internally implemented in at least one part of the body of a wearer of the wearable system.

In embodiments, the wearable system may be configured for use in users, subjects, or patients diagnosed with, known to suffer from, or displaying symptoms of a dermatological disorder. The dermatological disorder may include, for example, dry skin, eczema, erythema, and a burn, and combinations thereof.

In some embodiments, the environmental sensor may comprise at least one of a humidity sensor and a hydration sensor. In certain embodiments, the environmental sensor may further be configured to detect one or more characteristic features of an ambient area surrounding the wearer's skin. The one or more characteristic features of the ambient area may include, for example, a barometric air pressure of the ambient area, a humidity of the ambient area, an air temperature of the ambient area, an amount of radiation present in the ambient area, an amount of electromagnetic feedback present in the ambient area, and combinations thereof.

In an embodiment, the wearable system may comprise a transponder, transmitter, or transducer. The transponder, transmitter, or transducer may be coupled to the environmental sensor and configured to reflect and/or communicate information relating to the detected one or more characteristic features of the wearer's skin, as described above, to a reader as described herein. In some embodiments, the reader may comprise a hand-held device, a phone, a tablet computer, a portal, a garment, a wand, or combinations thereof. In certain embodiments, the transponder, transmitter, or transducer may be coupled to the wireless and battery-free environmental sensor via at least one wire, such that the transponder, transmitter, or transducer and the battery-free environmental sensor share one circuit. In some embodiments, the reader as described herein may be coupled to the transponder via a wireless connection as described herein.

In some embodiments, a kit may comprise a wearable system as described herein, and a product. In an embodiment, the product may comprise an active ingredient. In certain embodiments, the product may be, for example, an oral medication, an oral supplement, a topical medication, a topical composition, or combinations thereof. In other embodiments, the product may be any product described herein or known in the art. In certain embodiments, a kit may further comprise an environmental sensor configured to detect radiation incident on a wearer's skin.

While the present disclosure has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 sensors refers to groups having 1, 2, or 3 sensors. Similarly, a group having 1-5 sensors refers to groups having 1, 2, 3, 4, or 5 sensors, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1-38. (canceled)

39. A wearable system comprising:
a wireless and battery-free sensor configured to detect one or more characteristic features of a wearer's skin and communicate information relating to the one or more characteristic features of the wearer's skin to a reader;
wherein the reader is coupled to the wireless and battery-free sensor.

40. The wearable system of claim 39, wherein the reader is coupled to the wireless and battery-free sensor via a wireless connection.

41. The wearable system of claim 39, wherein the one or more characteristic features include a hydration level of the wearer's skin at one or more depths ranging from 100 nm to 20 mm under a surface of the wearer's skin.

42. The wearable system of claim 41, further comprising a controller configured to suggest performing at least one function in an event the hydration level deviates from a predetermined target value.

43. The wearable system of claim 42, wherein the at least one function comprises applying a product to the wearer's skin.

44. The wearable system of claim 43, wherein the product comprises at least one of a soap, a lotion, a gel, a spray, a dermatological product, and a combination thereof.

45. The wearable system of claim 42, wherein the controller is configured to suggest performing the at least one function by issuing a signal to the wearer by displaying at least some of the information relating to the detected one or more characteristic features of the wearer's skin.

46. The wearable system of claim 42, wherein the controller is coupled to an application software configured to receive the information relating to the one or more characteristic features of the wearer's skin, and to display the suggestion of performing the at least one function.

47. The wearable system of claim 39, further comprising an environmental sensor configured to detect radiation incident on the wearer's skin, wherein the environmental sensor comprises a dosimeter.

48. The wearable system of claim 47, wherein the environmental sensor is configured to detect radiations emitted at one or more wavelengths ranging from 180 nm to 1200 nm.

49. The wearable system of claim 39, wherein the wearable system is implemented in at least one of: a wearable garment, a bandage, a wound dressing, a pressure cuff, a hat, a shirt, a jacket, pants, a visor, shorts, a swimsuit, a hospital gown, a smock, hospital scrubs, a blouse, a dress, a skirt, a helmet, a glove, a mitten, an undergarment, footwear, eyewear, a tie, a necklace, an earring, a watch, a bracelet, a band, a hair accessory, a ring, a bag, a backpack, a belt, and a wearable accessory.

50. The wearable system of claim 39, wherein the wearable system is implemented in a location selected from the group consisting of: on a portion of a surface of the wearer's skin, and internally in at least one part of body of the wearer.

51. The wearable system of claim 39, wherein the wearable system is configured for use by wearers known to suffer from at least one of: dry skin, eczema, erythema, and a skin burn.

52. The wearable system of claim 39, wherein the wireless and battery-free sensor comprises at least one of a humidity sensor and a hydration sensor.

53. The wearable system of claim 39, wherein the wireless and battery-free sensor is further configured to detect one or more characteristic features of an ambient area surrounding the wearer's skin.

54. The wearable system of claim 39, wherein the one or more characteristic features of the ambient area include at least one of a barometric air pressure of the ambient area, a humidity of the ambient area, an ambient air temperature of the ambient area, an amount of radiation present in the ambient area, and an amount of electromagnetic feedback present in the ambient area.

55. A kit comprising the wearable system of claim 39, and a product selected from the group consisting of an oral medication, an oral supplement, a topical medication, a topical composition, a sunscreen, a soap, a lotion, a cream, a gel, a spray, and combinations thereof.

56. The kit of claim 55, further comprising an environmental sensor configured to detect radiation incident on a wearer's skin.

57. A wearable system comprising:
a wireless and battery-free sensor configured to:
detect one or more characteristic features of a wearer's skin;
communicate information relating to the one or more characteristic features of the wearer's skin to a reader; and
detect radiation incident on the wearer's skin;
wherein the reader is coupled to the wireless and battery-free sensor.

58. A kit comprising the wearable system of claim 57 and a product comprising at least one active ingredient, wherein the product is selected from the group consisting of an oral medication, an oral supplement, a topical medication, a topical composition, a sunscreen, a soap, a lotion, a cream, a gel, a spray, and combinations thereof.
